(19) United States
(12) Patent Application Publication
(10) Pub. No.: US 2021/0236032 A1
(43) Pub. Date: Aug. 5, 2021

(54) ROBOT-AIDED SYSTEM AND METHOD FOR DIAGNOSIS OF AUTISM SPECTRUM DISORDER

(71) Applicant: The George Washington University, Washington, DC (US)

(72) Inventors: Chung Hyuk PARK, Falls Church, VA (US); Hifza Javed, McLean, VA (US)

(21) Appl. No.: 17/159,691

(22) Filed: Jan. 27, 2021

Related U.S. Application Data
(60) Provisional application No. 62/967,873, filed on Jan. 30, 2020.

(51) Int. Cl. A61B 5/16; A61B 5/00; A61B 5/11; G16H 50/20; G16H 20/70

(52) U.S. Cl. CPC A61B 5/16; A61B 5/7275; A61B 5/1128; A61M 2021/0016; A61B 5/0077; G16H 50/20; G16H 20/70; A61B 5/7267

(57) ABSTRACT

The disclosed system uses facial expressions and upper body movement patterns to detect autism spectrum disorder. Emotionally expressive robots participate in sensory experiences by reacting to stimuli designed to resemble typical everyday experiences, such as uncontrolled sounds and light or tactile contact with different textures. The robot-child interactions elicit social engagement from the children, which is captured by a camera. A convolutional neural network, which has been trained to evaluate multimodal behavioral data collected during those robot-child interactions, identifies children that are at risk for autism spectrum disorder. Because the robot-assisted framework effectively engages the participants and models behaviors in ways that are easily interpreted by the participants, the disclosed system may also be used to teach children with autism spectrum disorder to communicate their feelings about discomforting sensory stimulation (as modeled by the robots) instead of allowing uncomfortable experiences to escalate into extreme negative reactions (e.g., tantrums or meltdowns).

### CROSS-REFERENCE TO RELATED APPLICATIONS

This application claims priority to U.S. Prov. Pat. Appl. No. 62/967,873, filed Jan. 30, 2020, which is hereby incorporated by reference.

### FEDERAL FUNDING

This system was made with government support from the National Institutes of Health (under Grant Number R01-HD082914, University Account No. 37987-1-CCLS29193F) and the National Science Foundation (under Grant No. 1846658, University Account No. 42008-1-CCLS29502F). The government has certain rights in the invention.

### BACKGROUND

Children with autism spectrum disorder typically experience difficulties in social communication and interaction. As a result, they display a number of distinctive behaviors including atypical facial expressions and repetitive behaviors such as hand flapping and rocking.

Sensory abnormalities are reported to be central to the autistic experience. Anecdotal accounts and clinical research both provide sufficient evidence to support this notion. One study found that, in a sample size of 200, over 90 percent of children with autism spectrum disorder had sensory abnormalities and showed symptoms in multiple sensory processing domains. The symptoms include hyposensitivity, hypersensitivity, multichannel receptivity, processing difficulties and sensory overload. A higher prevalence of unusual responses (particularly to tactile, auditory and visual stimuli) is seen in children with autism spectrum disorder when compared to their typically developing and developmentally delayed counterparts. The distress caused by some sensory stimuli can cause self-injurious and aggressive behaviors in children who may be unable to communicate their anguish. Families also report that difficulties with sensory processing and integration can restrict participation in everyday activities, resulting in social isolation for them and their child and impact social engagement.

Given the subjective, cumbersome and time intensive nature of the current methods of diagnosis, there is a need for a behavior-based approach to identify children at risk for autism spectrum disorder in order to streamline the standard diagnostic procedures and facilitate rapid detection and clinical prioritization of at-risk children. Children with autism spectrum disorder have been found to show a strong interest in technology in general and robots in particular. Therefore, robot-based tools may be particularly adept at stimulating socio-emotional engagement from children with autism spectrum disorder.

### SUMMARY

The disclosed system uses facial expressions and upper body movement patterns to detect autism spectrum disorder. For example, emotionally expressive robots may participate in sensory experiences by reacting to stimuli designed to resemble typical everyday experiences, such as uncontrolled sounds and light or tactile contact with different textures. The robot-child interactions elicit social engagement from the children, which is captured by a camera. A convolutional neural network, which has been trained to evaluate multimodal behavioral data collected during those robot-child interactions, identifies children that are at risk for autism spectrum disorder.

The disclosed system has been shown to accurately identify children at risk for autism spectrum disorder. Meanwhile, the robot-assisted framework effectively engages the participants and models behaviors in ways that are easily interpreted by the participants. Therefore, with long-term exposure to the robots in this setting, the disclosed system may also be used to teach children with autism spectrum disorder to communicate their feelings about discomforting sensory stimulation (as modeled by the robots) instead of allowing uncomfortable experiences to escalate into extreme negative reactions (e.g., tantrums or meltdowns).

### BRIEF DESCRIPTION OF THE DRAWINGS

The accompanying drawings are incorporated in and constitute a part of this specification. It is to be understood that the drawings illustrate only some examples of the disclosure and other examples or combinations of various examples that are not specifically illustrated in the figures may still fall within the scope of this disclosure. Examples will now be described with additional detail through the use of the drawings.

FIG. 1 is a diagram of a robot-aided platform according to an exemplary embodiment.

FIG. 2 illustrates example emotions expressed by the humanoid robot according to an exemplary embodiment.

FIG. 3 illustrates example emotions expressed by the facially expressive robot according to an exemplary embodiment.

FIG. 4 illustrates sensory stations according to an exemplary embodiment.

FIG. 5 illustrates the facial keypoints and body tracking keypoints extracted according to an exemplary embodiment.

FIG. 6 is a diagram illustrating the convolutional neural network according to an exemplary embodiment.

FIG. 7 illustrates a graph 700 depicting the engagement of one participant using the disclosed system according to an exemplary embodiment.

FIG. 8 illustrates graphs of each target behavior during an interaction with each emotionally expressive robot.

### DETAILED DESCRIPTION

In describing the illustrative, non-limiting embodiments illustrated in the drawings, specific terminology will be resorted to for the sake of clarity. However, the disclosure is not intended to be limited to the specific terms so selected, and it is to be understood that each specific term includes all technical equivalents that operate in similar manner to accomplish a similar purpose. Several embodiments are described for illustrative purposes, it being understood that the description and claims are not limited to the illustrated embodiments and other embodiments not specifically shown in the drawings may also be within the scope of this disclosure.

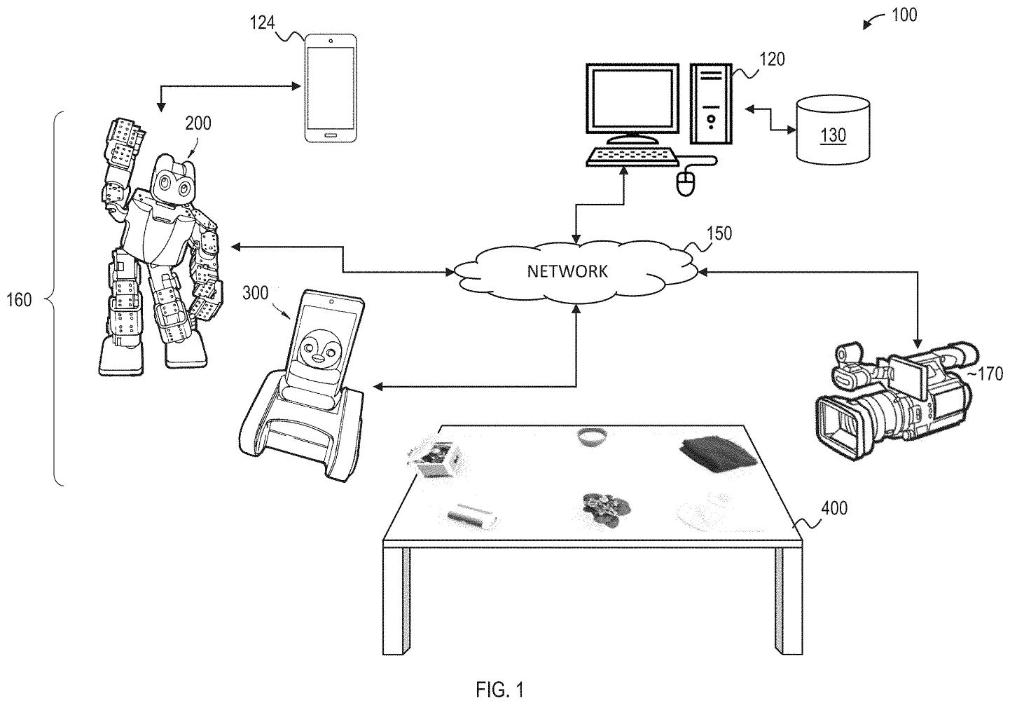

FIG. 1 is a diagram of a robot-aided platform 100 according to an exemplary embodiment.

As shown in FIG. 1, the platform 100 may include a computer 120, a database 130, one or more networks 150, one or more emotionally expressive robots 160, a video camera 170, and a number of sensory stations 400. The one or more emotionally expressive robots 160 may include, for example, a humanoid robot 200 and a facially expressive robot 300.

The computer 120 may be any suitable computing device programmed to perform the functions described herein. The computer 120 includes at least one hardware processor and memory (i.e., non-transitory computer readable storage media). For example, the computer 120 may be a server, a personal computer, etc.

The network(s) 150 may include a local area network, the Internet, etc. The computer 120, the emotionally expressive robot(s) 160 and the video camera 170 may communicate via the network(s) 150 using wired or wireless connections (e.g., ethernet, WiFi, etc.).

The emotionally expressive robot(s) 160, which are described in detail below, may be controllable via the computer 120. Alternatively, an emotionally expressive robot 160 may be controllable via a computing device 124 (e.g., a smartphone, a tablet computer, etc.), for example via wireless communications (e.g., Bluetooth).

The video camera 170 may be any suitable device configured to capture and record video images. For example, the video camera 170 may be a digital camcorder, a smartphone, etc. The video camera 170 may be configured to transfer those video images to the computer 120 via the network(s) 150. However, as one of ordinary skill in the art would recognize, those video images may be stored by the video camera 170 and transferred to the computer 120, for example via a wired connection or physical storage medium.

The humanoid robot 200 may include a torso, arms, legs, and a face. The humanoid robot 200 may be programmable such that it mimics the expression of human emotion through gestures, speech, and/or facial expressions. The humanoid robot 200 may be a Robotis Mini available from Robotis, Inc.

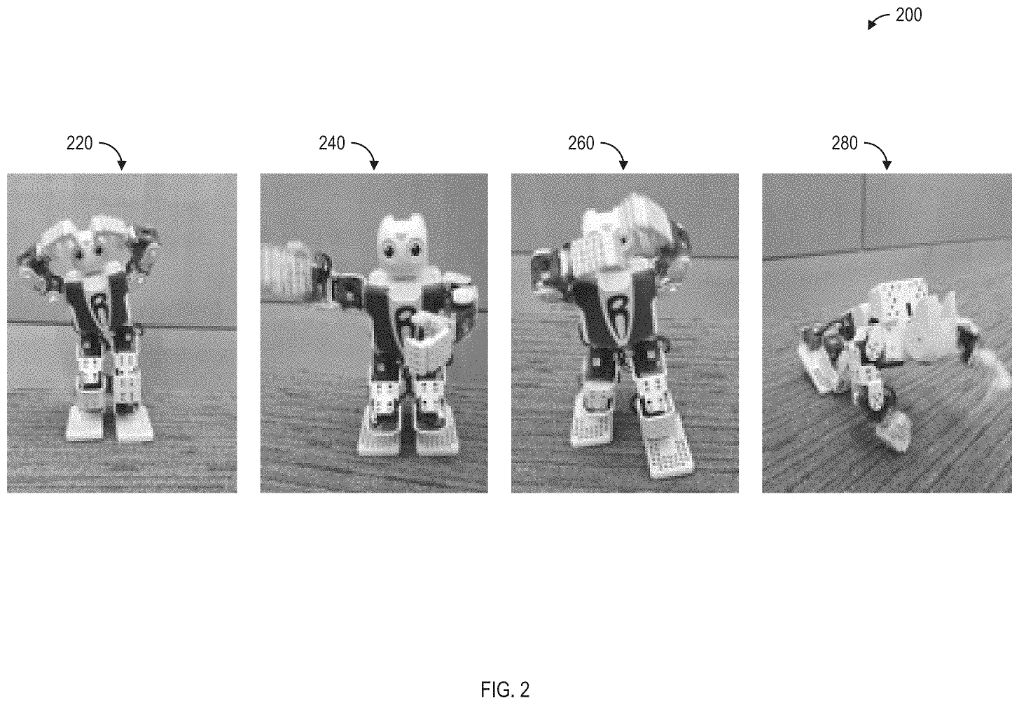

FIG. 2 illustrates example emotions expressed by the humanoid robot 200 according to an exemplary embodiment.

The humanoid robot 200 may be programmed to portray the emotions that are commonly held to be the six basic human emotions (happiness, sadness, fear, anger, surprise and disgust) as well as additional emotional states relevant to interactions involving sensory stimulation. As shown in FIG. 2, the humanoid robot 200 may be programmed to portray emotions such as dizzy 320, happy 340, scared 360, and frustrated 380. Additionally, the humanoid robot 200 may be programmed to portray additional emotions and physical states (not pictured), including unhappy, sniff, sneeze, excited, curious, wanting, celebrating, bored, sleepy, sad, nervous, tired, disgust, crying, and/or angry.

The facially expressive robot 300 may include a wheeled platform and a display (e.g., a smartphone display). The facially expressive robot 300 may be programmable such that it mimics the expression of human emotion through motion, sound effects, and/or facial expressions. The facially expressive robot 300 may be a Romo, a controllable, wheeled platform for an iPhone that was previously available from Romotive Inc.

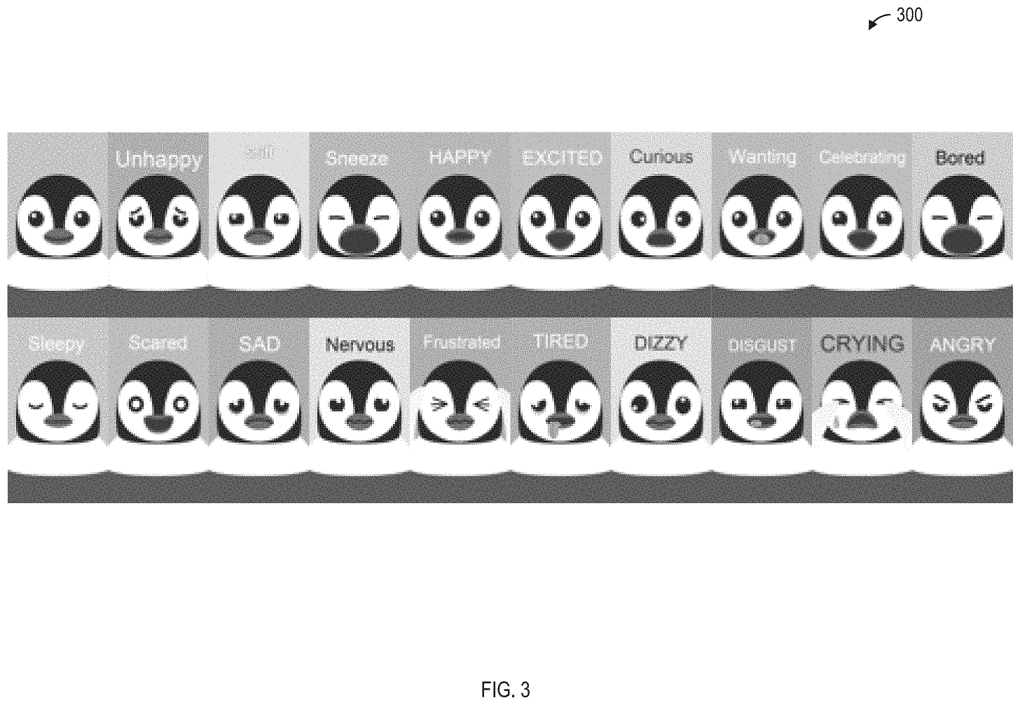

FIG. 3 illustrates example emotions expressed by the facially expressive robot 300 according to an exemplary embodiment. In the example shown in FIG. 3, the facially expressive robot 300 is programmed to display an animation that includes a custom-designed penguin avatar.

Similar to the humanoid robot 200, the facially expressive robot 300 may be programmed to portray the emotions that are commonly held to be the six basic human emotions (happiness, sadness, fear, anger, surprise and disgust) as well as additional emotional states relevant to interactions involving sensory stimulation. As shown in FIG. 3, the facially expressive robot 300 may be programmed to display animations that portray emotions (and physical states) that include neutral, unhappy, sniff, sneeze, happy, excited, curious, wanting, celebrating, bored, sleepy, scared, sad, nervous, frustrated, tired, dizzy, disgust, crying, and/or angry. Each animation for each emotion or physical state may be accompanied by a dedicated background color, complementary changes in the tilt angle of the display, and/or movement of the facially expressive robot 300 (e.g., circular or back-and-forth movement of the treads).

In either or both instances, the emotionally expressive robot(s) 160 may be programmed to depict simple but meaningful behaviors, combining all available modalities of emotional expression (e.g., movement, speech and facial expressions). The emotionally expressive robot(s) 160 may be designed to be expressive, clear and straightforward so as to facilitate interpretation in the context of the scenario being presented at the given sensory station 400 (discussed below). A humanoid robot 200 that communicates through gestures and speech is capable of responding to the sensory stimulation in a manner that resembles natural human-human communication. According, the humanoid robot 200 is capable of meaningfully responding to sensory stimulation without acting out explicit emotions. By contrast, a facially expressive robot 300 may use relatively primitive means of communication, like facial expressions, sound effects and movements. Therefore, the facially expressive robot 300 may be programmed to react to sensory stimulation through explicit emotional expressions joined one after another to form meaningful responses.

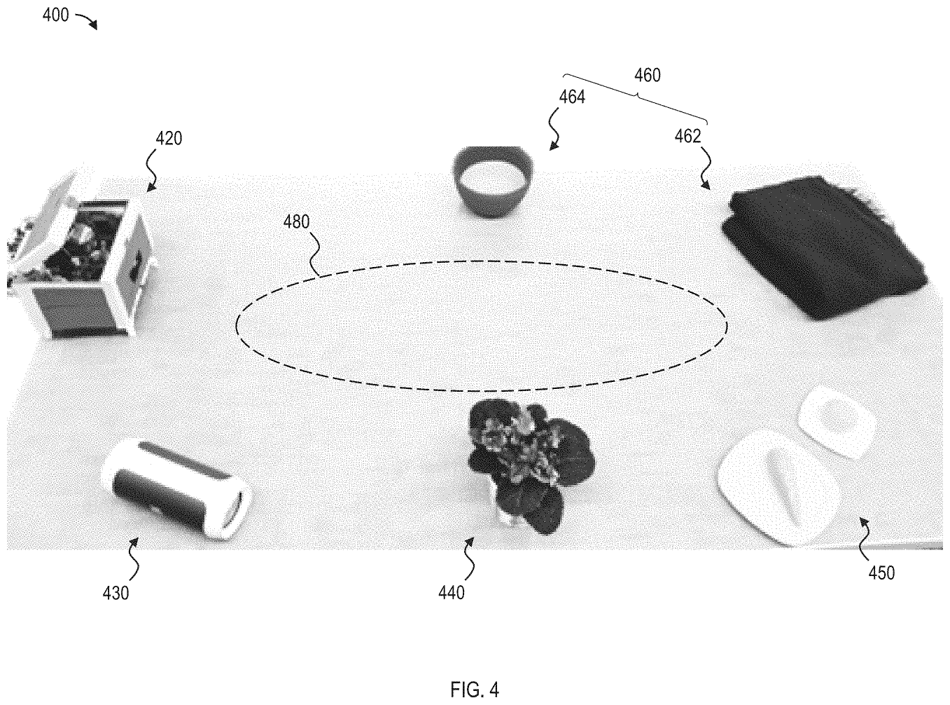

FIG. 4 illustrates the sensory stations 400 according to an exemplary embodiment.

As shown in FIG. 4, the sensory stations 400 may include a seeing station 420, a hearing station 430, a smelling station 440, a tasting station 450, a touching station 460, and a celebration station 480. The sensory stations 400 are designed to resemble real world scenarios that form a typical part of one's everyday experiences, such as uncontrolled sounds and light in a public space (e.g., a mall or a park) or tactile contact with clothing made of fabrics with different textures. The emotionally expressive robot(s) 160 are programmed to interact with each sensory station 400 and react in a manner that demonstrates socially acceptable responses to each stimulation. The emotionally expressive robot(s) 160 interact with each sensory station 400 in a manner that is interactive and inclusive of the child, such that the emotionally expressive robot 160 and the child engage in a shared sensory experience.

The seeing station 420 may designed to provide visual stimulus. For example, the seeing station 420 may include a flashlight inside a lidded box (e.g., constructed from a LEGO Mindstorm EV3 kit) with an infrared sensor that opens the lid of the box when movement is detected in proximity. The emotionally expressive robot 160 may be programmed to move toward the seeing station 420 at which point the lid of the box is opened and the flashlight directs a bright beam of light in the direction of the approaching emotionally expressive robot 160.

The hearing station 430 may be designed to provide an auditory stimulus. For example, the hearing station 430 may include a Bluetooth speaker play plays music. The smelling station 440 may be designed to provide olfactory stimulus. For example, the smelling station 440 may include scented artificial flowers inside a flowerpot. The tasting station 450 may be designed to provide gustatory stimulus. For example, the tasting station 450 may include two small plastic plates with two different food items. (Those food items may be modified according to likes and dislikes of each every subject child.) The touching station 460 may be designed to provide tactile stimulus. For example, the touching station may include a soft blanket 462 and a bowl of sand 464 (e.g., with golden stars hidden inside it).

Each of the emotionally expressive robot(s) 160 may be programmed to travel (e.g., walk and/or drive) to each sensory station 400 and interact with the sensory stimuli presented at each sensory station 400. While interacting with each sensory stimuli, the emotionally expressive robot(s) 160 may be programmed to initiate a conversation with the child and facilitate a joint sensory experience.

### Diagnosis

The video camera 170 records each interaction between each child and the emotionally expressive robot(s) 160. Images of each child are then analyzed by the computer 120.

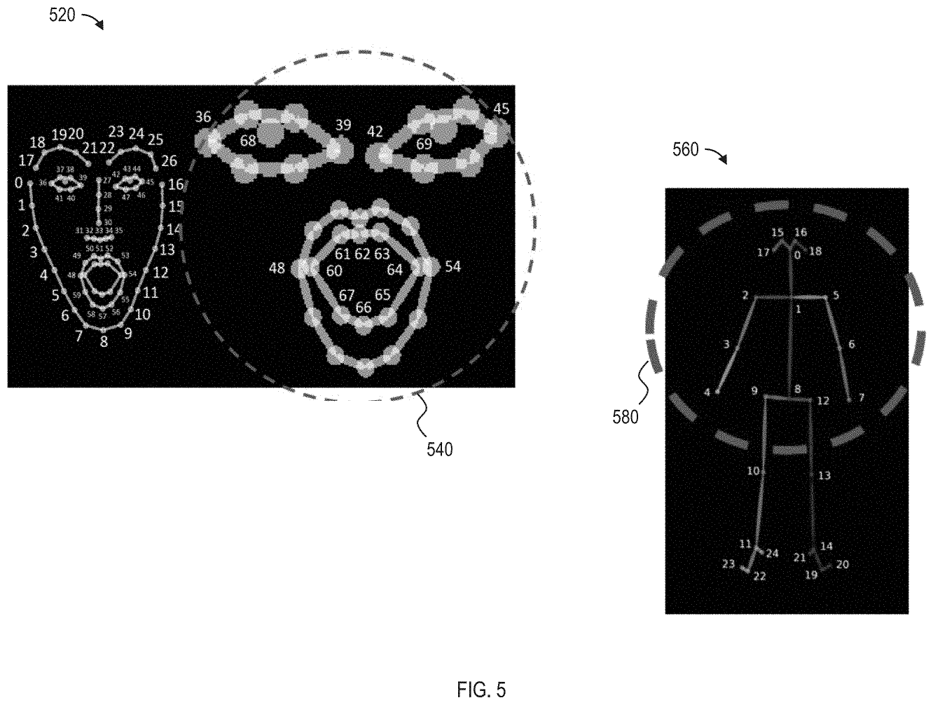

FIG. 5 illustrates facial keypoints 520 and body tracking keypoints 560 according to an exemplary embodiment. In image analysis, “keypoints” are distinctive points in an input image that are invariant to rotation, scale and distortion. Facial keypoints 520, sometimes referred to as “facial landmarks,” are specific areas of the face (e.g., nose, eyes, mouth, etc.) identified in images of faces. Similarly, body tracking keypoints 560 are specific points of the bodies identified in images of people. Facial keypoints 520 and body tracking keypoints 560 are identified in images in order to identify the coordinates of the specified body part. Image recognition systems generally use the facial keypoints 520 to perform facial recognition, emotion recognition, etc. Similarly, body tracking keypoints 560 may be used to identify body poses and movements.

Body tracking keypoints 560 and facial keypoints 520 are extracted from the video images by the computer 120, for example using OpenPose. As shown in FIG. 5, for example, the computer 120 may analyze a subset 540 of the facial keypoints 520 originating from the nose and eyes. Additionally, because the children may interact with the sensory stations 400 from behind a table, the computer 120 may extract only upper body keypoints 580 originating from the arms, torso, and head of the child.

The computer 120 may derive movement features from the body upper body keypoints 580, for example using Laban movement analysis, to determine the intent behind human movement. In machine learning, pattern recognition, and image processing, “feature extraction” starts from an initial set of measured data and builds derived values (“features”) intended to be informative and non-redundant, facilitating the subsequent learning and generalization steps. As described below, those movement features derived by the computer 120 may include weight, space, and time. Those movement features may be derived using a moving time window (e.g., a 1 second window) to capture the temporal nature of the data. The three derived movement features may be combined with facial keypoints (e.g., 68 facial keypoints originating from the nose and eyes) to form a dataset. Accordingly, the dataset may include a total of 71 features.

As mentioned above, the computer may derive movement features from the upper body keypoints 580. Those movement features may include weight, space, and time.

Weight can be described as the intensity of perceived force in movement. High and constant intensity is considered high weight (strong) and the opposite is considered low weight (light). Strong weight characterizes bold, forceful, powerful, and/or determined intention. Light weight characterizes delicate, sensitive buoyant, and easy intention. Weight may be derived by the computer 120 as follows:

$$Weight = {\sum }_{i} {τ}_{i} {ω}_{i} ( t )$$

where:

$${τ}_{i} = F * L = {L}^{2} {ω}_{i}^{2} sin ( θ ) * mas s$$

$${ω}_{i} = \frac{d θ}{d t}$$

$$i = Joint Number$$

Space is a measure of the distance of the legs and arms to the body. Space is considered low (direct) when legs and arms are constantly close to the body center and is considered high (indirect) if a person is constantly using outstretched movements. Direct space is characterized by linear actions, focused and specific actions, and/or attention to a singular spatial possibility. Indirect space characterizes flexibility of the joints, three-dimensionality of space, and/or all-around awareness. Because the disclosed system may be limited to analyzing upper body keypoints 580, space may be indicative of the distance of the arms of the child relative to the body of the child. Space may be derived by the computer 120 as follows:

Space=(0.5| [symbol] || [symbol] |sin(θ₁))+(0.5| [symbol] || [symbol] |sin(θ₂))

where
[symbol] =Left Shoulder to Left Hand
[symbol] =Right Shoulder to Left Shoulder
[symbol] =Right Hand to Right Shoulder
[symbol] =Left Hand to Right Hand
θ₁=Angle between \vec{a} & \vec{d}
θ₂=Angle between \vec{c} & \vec{b}

Time is a measure of the distinct change from one prevailing tempo to some other tempo. Space is considered high when movements are sudden and low when movements are sustained. Sudden movements are characterized as unexpected, isolated, surprising, and/or urgent. Sustained movements are characterized as continuous, lingering, indulging in time, and/or leisurely. Time may be calculated by the computer 120 as follows:

$$T i m {e}_{i} = {\sum }_{i} {\dot{ω}}_{i} ( t )$$

where:

\dot{ω}ᵢ=Angular Velocity for Joint i

As described above, preferred embodiments utilize a video camera 170 to capture video images of children and a computer 120 to extract facial keypoints 520 and body tracking keypoints 560 and derive movement features of those children. However, the disclosed system is not limited to a video camera 170 and may instead utilize any sensor (e.g., RADAR, SONAR, LIDAR, etc.) suitably configured to capture data indicative of the facial keypoints 520 and body tracking keypoints 560 of the child over time.

Referring back to FIG. 1, the subset 540 of the facial keypoints 520 and the movement features (e.g., weight, space, and time) are stored in the database 130. Meanwhile, the computer 120 includes a convolutional neural network 600 designed to process that data and identify children at risk for autism spectrum disorder.

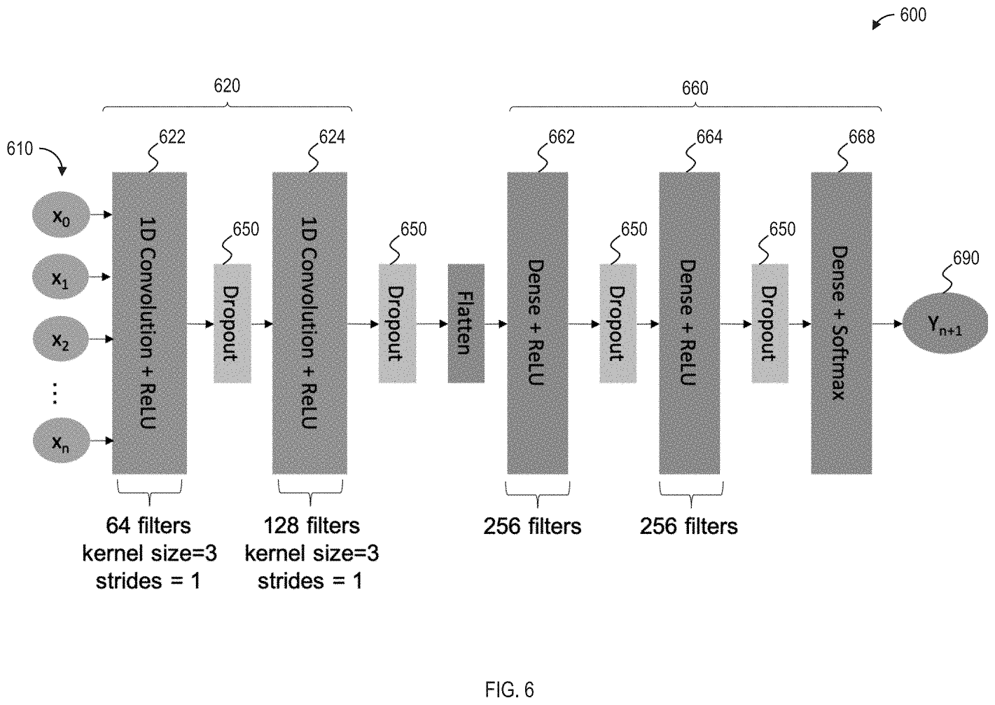

FIG. 6 is a diagram illustrating the convolutional neural network 600 according to an exemplary embodiment.

As shown in FIG. 6, convolutional neural network 600 may include two Conv1D layers (1-dimensional convolution layers) 620 to identify temporal data patterns, three dense layers 660 for classification, and multiple dropout layers 650 to avoid overfitting.

The Conv1D layers 620 may include a first Conv1D layer 622 and a second Conv1D layer 624. The first Conv1D layer 622 may include five channels with 64 filters and the second Conv1D layer 624 may include 128 filters. Each of the Conv1D layers 620 may have a kernel size of 3. The convolutional neural network 600 may include two Conv1D layers 620 to extract high-level features from the temporal data because the dataset being used has a high input dimension and a relatively small number of datapoints.

Each dropout layer 650 may have a dropout rate of 20 percent.

The dense layers 660 may include a first dense layer 662, a second dense layer 664, and a third dense layer 668. Since the data have a non-linear structure, the first dense layer 662 and the second dense layer 664 may be used to spread the feature dimension while the third dense layer 668 generates an output dimension 690.

The convolutional neural network 600 models the risk of autism spectrum disorder as a binary classification problem. The convolutional neural network 600 is trained using a corpus of data captured by the disclosed system analyzing children that have been diagnosed with autism spectrum disorder and children having been diagnosed as not at risk for autism spectrum disorder (e.g., typically developing). The convolutional neural network 600 can then be supplied with input data 610, for example the facial keypoints 520 and the movement features (e.g., weight, space, and time) described above. Having been trained on a dataset characterizing children of known risk, the convolutional neural network 600 is then configured to generate an output dimension 690 indicative of the subject's risk for autism spectrum disorder.

The disclosed system has been shown to accurately identify children at risk for autism spectrum disorder. In an initial study, the convolutional neural network 600 was trained on 80 percent of the interaction data and the remaining 20 percent were used to validate its performance. The convolutional neural network 600 achieved high accuracy (0.8846), precision (0.8912), and recall (0.8853).

Unlike previous methods, the disclosed system identifies children at risk for autism spectrum disorder based only on behavioral data captured through video recordings of a naturalistic interaction with social robots. The movement of the child was not restricted and no obtrusive sensors were used. Accordingly, the disclosed system and method can easily be generalized to other interactions (e.g., play time at home) increasing the utility of the disclosed method. The possibility of using the disclosed system in additional settings also raises the possibility that larger datasets may be obtained, thereby increasing the accuracy of the disclosed method.

### Treatment

As described above, the sensory stations 400 closely resemble situations that children would encounter frequently in their everyday lives. Therefore, they are relatable and easy to interpret. Given the strong interest in technology from children with autism spectrum disorder, the emotionally expressive robot(s) 160 may be used to elicit a higher level of socio-emotional engagement from these children. For example, the emotionally expressive robot(s) 160 navigating the sensory stations 400 may be used to demonstrate socially acceptable responses to stimulation and encourage children to become more receptive to a variety of sensory experiences and to effectively communicate their feelings if the experiences cause them discomfort.

The emotionally expressive robot(s) 160 may be programmed to show both positive and negative responses at some of the sensory stations 400 with the aim of demonstrating to the children how to communicate their feelings even when experiencing discomforting or unfavorable sensory stimulation (instead of allowing the negative experience to escalate into a tantrum or meltdown). The negative reactions may be designed not to be too extreme so as to focus on the communication of one's feelings rather than encouraging intolerance of the stimulation.

At the seeing station 420, the emotionally expressive robot(s) 160 may be programmed to demonstrate effectively handle uncomfortable visual stimuli and to communicate discomfort instead of allowing it to manifest as extreme negative reactions (tantrums/meltdowns). This can be especially useful in controlled environments like movie theaters and malls where light intensity cannot be fully regulated.

The hearing station 430 may improve tolerance for sounds louder than those to which one is accustomed, to learn to not be overwhelmed by music, and to promote gross motor movements by encouraging dancing along to it. This can be especially useful in uncontrolled environments like movie theaters and malls where sounds cannot be fully regulated.

At the smelling station 440, the emotionally expressive robot(s) 160 may be programmed to not react with extreme aversion to odors that may be disliked and to communicate the dislike instead. This can be useful for parents of children with autism spectrum disorder who are very particular about the smell of their food, clothes, and/or environments etc.

At the tasting station 450, the emotionally expressive robot(s) 160 may be programmed to demonstrate diversifying one's food preferences instead of adhering strictly to the same ones.

At the touching station 460, the emotionally expressive robot(s) 160 may be programmed to demonstrate acclimating oneself to different textures by engaging in tactile interactions with different materials. This is especially useful for those children with autism spectrum disorder who may be sensitive to the texture of their clothing fabrics and/or those who experience significant discomfort with wearables (e.g., hats, wrist watches, etc.).

At the celebration station 480, the emotionally expressive robot(s) 160 may be programmed to convey a sense of shared achievement while also encouraging the children to practice their motor and vestibular skills by imitating the celebration routines of the robots.

The emotionally expressive robot(s) 160 may be particularly effective after the children have already interacted with the emotionally expressive robot(s) 160 over several sessions. Once an emotionally expressive robot 160 has formed a rapport with the child by liking and disliking the same foods as the child, for example, it could start to deviate from those responses and encourage the child to be more receptive to the foods their robot “friends” prefer. To achieve this goal, for example, different food items may be introduced in the tasting station 450 in the future sessions.

While the disclosed system may include any emotionally expressive robot 160, the humanoid robot 200 and the facially expressive robot 300 are examples of preferred emotionally expressive robots 160 for a number of reasons. The emotionally expressive robot(s) 160 are preferably not be too large in size in order to prevent children from being intimidated by them. The emotionally expressive robot(s) 160 are preferably capable of expressing emotions through different modalities such as facial expressions, gestures and speech. The emotionally expressive robot(s) 160 are preferably friendly in order to form a rapport with the children.

The sensory stations 400 are preferably designed to be relatable to the children such that they are able to draw the connection between the stimulation presented to the emotionally expressive robot(s) 160 and that experienced by them in their everyday lives. The activity being conducted is preferably able to maintain a child's interest through the entire length of the interaction. Accordingly, the content (and duration) of the activity is preferably appealing to the children.

The actions performed by the emotionally expressive robot(s) 160 is preferably simple and easy to understand for children in the target age range. The gestures, speech, facial expressions and/or body language emotionally expressive robot(s) 160 is preferably combined to form meaningful and easily interpretable behaviors. The emotion library of the emotionally expressive robot(s) 160 is preferably large enough to effectively convey different reactions to the stimulation but also simple enough to be easily understood by the children.

In order to derive a meaningful quantitative measure of engagement, we utilized several key behavioral traits of social interactions, including gaze focus, vocalizations and verbalizations, smile, triadic interactions, self-initiated interactions and imitation:

| Behavior | Description |
|---|---|
| Eye gaze focus | Deficits in social attention and establishing eye |
| | contact are two of the most commonly reported |
| | deficits in children with autism spectrum disorder. |
| | We therefore used the children's gaze focus on the |
| | robots and/or the setup to mark the presence of this |
| | behavior. |
| Vocalizations/ | The volubility of utterances produced by children |
| verbalizations | with autism spectrum disorder is low compared to |
| | their typically developing counterparts. Since |
| | communication is a core aspect of social |
| | responsiveness, the frequency and duration of the |
| | vocalizations and verbalizations produced by the |
| | children during the interaction is also important in |
| | computing the engagement index. |
| Smile | Smiling has also been established as an aspect of |
| | social responsiveness. We recorded the frequency |
| | and duration of smiles displayed by the children |
| | while interacting with the robots, as a contributing |
| | factor to the engagement index. |
| Triadic | A triadic relationship involves three agents, including |
| interactions | the child, the robot and a third person that may be the |
| | parent or the instructor. In this study, the robot acts as |
| | tool to elicit interactions between the child and other |
| | humans. An example of such interactions is the |
| | child sharing her excitement about the dancing robot |
| | by directing the parent's attention to it. |
| Self-initiated | Children with autism spectrum disorder prefer to |
| interactions | play alone and make fewer social initiations compared |
| | to their peers. Therefore, we recorded the frequency |
| | and duration of the interactions with the robot initiated |
| | by the children as factors contributing to the |
| | engagement index. Examples of self-initiated |
| | interactions can include talking to the robots, |
| | attempting to feed the robots, guiding the robots to |
| | the next station etc. without any prompts from the |
| | instructors. |
| Imitation | Infants have been found to produce and recognize |
| | imitation from the early stages of development, and |
| | both these skills have been linked to the development |
| | of socio-communicative abilities. In this study, we |
| | monitored a child's unprompted imitation of the |
| | robot behaviors as a measure of their engagement |
| | in the interaction. |

The aforementioned behaviors were selected because they have proven to be useful measures of social attention and social responsiveness from previous studies.

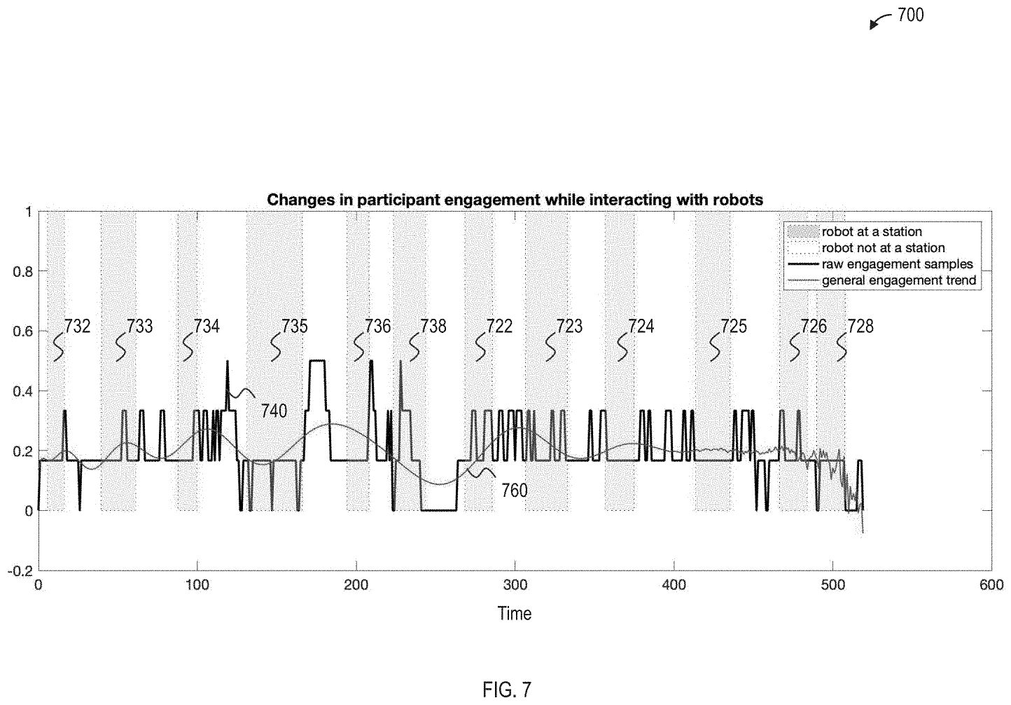

FIG. 7 illustrates a graph 700 depicting the engagement of one participant using the disclosed system according to an exemplary embodiment.

As shown in FIG. 7, the graph includes an engagement index 740 and a general engagement trend 760. Video data was coded for the target behaviors above (smile, eye gaze focus, vocalizations/verbalizations, triadic interaction, self-initiated interaction, and imitation) and the engagement index 740 was derived as the indicator of every child's varying social engagement throughout the interaction with the emotionally expressive robots 160. The engagement index 740 was computed as a sum of these factors, each with the same weight, such that the maximum value of the engagement index 740 was 1.

Each behavior contributed a factor of ⅙ to the engagement index 740. For example, for a participant observed to have a smile and gaze focus while interacting with the humanoid robot 200 during the tasting station 450 but only gaze focus following the end of the station, the engagement index 740 was assigned a constant value of ⅙+⅙=⅓ for the entire duration of the station, and reduced to ⅙ immediately after its end. Any changes in engagement within an interval of 1 second were detected and reflected in the engagement index 740.

Time periods when each emotionally expressive robot 160 interacts with each sensory station 400, including time period 732, when the facially expressive robot 300 interacted with the seeing station 420; time period 733, when the facially expressive robot 300 interacted with the hearing station 430; including time period 734, when the facially expressive robot 300 interacted with the smelling station 440; including time period 735, when the facially expressive robot 300 interacted with the tasting station 450; including time period 736, when the facially expressive robot 300 interacted with the touching station 460; including time period 738, when the facially expressive robot 300 interacted with the celebration station 480; time period 722, when the humanoid robot 200 interacted with the seeing station 420; time period 723, when the humanoid robot 200 interacted with the hearing station 430; including time period 724, when the humanoid robot 200 interacted with the smelling station 440; including time period 725, when the humanoid robot 200 interacted with the tasting station 450; including time period 726, when the humanoid robot 200 interacted with the touching station 460; and including time period 728, when the humanoid robot 200 interacted with the celebration station 480.

Analyzing the engagement index 740 when each emotionally expressive robot 160 interacts with each sensory station 400 allows for a comparison of the effectiveness of each sensory station 400 in eliciting social engagement from the participants.

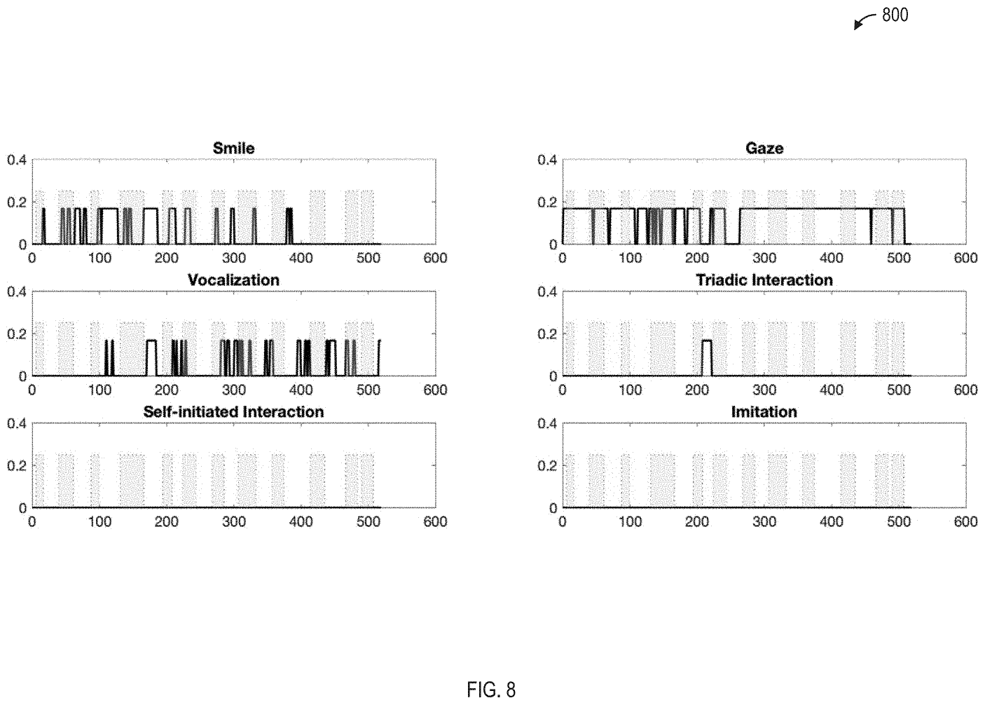

FIG. 8 illustrates graphs 800 of each target behavior (smile, eye gaze focus, vocalizations/verbalizations, triadic interaction, self-initiated interaction, and imitation) during an interaction with each emotionally expressive robot 160 according to an exemplary embodiment. Labels for each time period 732, 733, etc. are omitted for clarity, but they are legibility but are the same as shown in FIG. 7. By identifying the target behaviors elicited by each emotionally expressive robot 160 at each sensory station 400, the frequency each target behavior and the sensory stations 400 emotionally expressive robot 160 responsible for eliciting them can be compared.

Finally, the engagement generated by each emotionally expressive robot 160 may also be assessed individually and compared to study the social engagement potential of each emotionally expressive robots 160 in this sensory setting.

Using the method to derive the engagement index 740 described above, several other metrics were also generated to evaluate various aspects of the disclosed system. First, the session comprising interactions with both emotionally expressive robots 160 was analyzed as a whole, resulting in consolidated engagement metrics. In addition, engagement resulting from each target behavior was also computed to study the contribution of each target behavior toward the engagement index. As an example, an engagement metric resulting from the vocalizations of participant X was computed as:

$${eng}_{voc , X} = \frac{\begin{matrix}sum of all vocalization factors \\ throughout the session\end{matrix}}{\begin{matrix}sum of engagement factors from all \\ target behaviors throughout the session\end{matrix}}$$

By isolating the engagement resulting from each emotionally expressive robot 160, the metrics generated by the humanoid robot 200 and the facially expressive robot 300 may be compared to evaluate the impact of each emotionally expressive robot 160. Once again, an overall engagement index was obtained for each emotionally expressive robot 160 as an indicator of its performance throughout its interaction in addition to a breakdown in terms of the target behaviors that comprise the engagement. The engagement metric for the interaction of participant X with the facially expressive robot 300 (“Romo”) was calculated as:

$${eng}_{Romo , X} = \frac{\begin{matrix}sum of all engagement factors \\ throughout interaction with Romo\end{matrix}}{\begin{matrix}sum of engagement factors throughout \\ session with both robots\end{matrix}}$$

Similarly, the engagement metric resulting from the vocalizations of participant X while interacting with the facially expressive robot 300 (“Romo”) was calculated as:

$${eng}_{Romo , voc , X} = \frac{\begin{matrix}sum of all vocalization factors \\ throughout interaction with Romo\end{matrix}}{\begin{matrix}sum of engagement factors throughout \\ session with both robots\end{matrix}}$$

An analysis was then performed to study the differences in engagement at each sensory station 400. This was analyzed separately for each emotionally expressive robot 160 so as to derive an understanding of the engagement potential of each station per robot. The engagement metric resulting from the hearing station 430 while participant X interacted with the humanoid robot 200 (“Mini”) was calculated as:

$${eng}_{Mini , hear , X} = \frac{\begin{matrix}sum of all engagement factors at hearing \\ station during interaction with Mini\end{matrix}}{\begin{matrix}sum of engagement factors throughout \\ session with Mini\end{matrix}}$$

In addition, a breakdown of engagement at each sensory station 400 was obtained in terms of the elicited target behaviors and analyzed separately for each emotionally expressive robot 160. This allowed for a finer-grain assessment of the capability of each sensory station 400 for eliciting the individual target behaviors. For example, the engagement metric resulting from the gaze of participant X at the smelling station 440 while interacting with the humanoid robot 200 (“Mini”) was calculated as:

$${eng}_{Mini , smell , gaze , X} = \frac{\begin{matrix}sum of all gaze factors at the smelling \\ station with Mini\end{matrix}}{\begin{matrix}sum of engagement factors at \\ smelling station with Mini\end{matrix}}$$

The aforementioned metrics enabled each sensory station 400 and each emotionally expressive robot 160 to be evaluated to achieve a comprehensive understanding of the potential of the disclosed system and identify areas requiring further improvement.

The drawings may illustrate—and the description and claims may use—several geometric or relational terms and directional or positioning terms, such as upper. Those terms are merely for convenience to facilitate the description based on the embodiments shown in the figures and are not intended to limit the invention. Thus, it should be recognized that the invention can be described in other ways without those geometric, relational, directional or positioning terms. And, other suitable geometries and relationships can be provided without departing from the spirit and scope of the invention.

The foregoing description and drawings should be considered as illustrative only of the principles of the disclosure, which may be configured in a variety of shapes and sizes and is not intended to be limited by the embodiment herein described. Numerous applications of the disclosure will readily occur to those skilled in the art. Therefore, it is not desired to limit the disclosure to the specific examples disclosed or the exact construction and operation shown and described. Rather, all suitable modifications and equivalents may be resorted to, falling within the scope of the disclosure.

What is claimed is:

1. A system for determining whether a child is at risk for autism spectrum disorder based on movement and facial expression, the system comprising:
a video camera that captures video images of the child;
a computer that:
extracts body tracking keypoints and facial keypoints from the video images; and
derives movement features from the body tracking keypoints; and
a convolutional neural network, trained on a dataset that includes movement features and facial keypoints of children diagnosed with autism spectrum disorder, that:
receives the movement features derived from the video images and the facial keypoints extracted from the video images; and
generates a diagnosis indicative of the risk for autism spectrum disorder based on the facial keypoints extracted from the video images of the child and the movement features derived from the video images of the child.

2. The system of claim 1, wherein the movement features include a weight feature indicative of intensity of perceived force in the movement, a space feature indicative of distance of the arms of the child relative to the body of the child, and a time feature indicative of a change in tempo in the movement.

3. The system of claim 1, wherein the convolutional neural network includes two one-dimensional convolution layers to identify temporal data patterns, three dense layers for classification, and a plurality of dropout layers to avoid overfitting.

4. The system of claim 1, further comprising an emotionally expressive robot programmed to mimic the expression of human emotion.

5. The system of claim 4, wherein the emotionally expressive robot comprises a humanoid robot programmed to mimic the expression of human emotion through gestures or speech.

6. The system of claim 4, wherein the emotionally expressive robot comprises a facially expressive robot programmed to mimic the expression of human emotion through facial expression.

7. The system of claim 4, wherein the video camera captures video images of the child interacting with the emotionally expressive robot.

8. The system of claim 4, further comprising a plurality of sensory stations that each provide sensory stimulation.

9. The system of claim 8, wherein the plurality of sensory stations include a seeing station that provides visual stimulus, a hearing station that provides auditory stimulus, a smelling station provide olfactory stimulus, a tasting station that provides gustatory stimulus, or a touching station that provides tactile stimulus.

10. The system of claim 8, wherein the video camera captures video images of the child observing the emotionally expressive robot interacting with each of the sensory stations.

11. A method for determining whether a child may be at risk for autism spectrum disorder based on movement and facial expression, the method comprising:
receiving video images of the child by a computer;
extracting body tracking keypoints and facial keypoints from the video images by the computer;
deriving movement features from the body tracking keypoints by the computer;
providing the movement features derived from the video images and the facial keypoints extracted from the video images, by the computer, to a convolutional neural network trained on a dataset that includes movement features and facial keypoints of children diagnosed with autism spectrum disorder; and
generating a diagnosis indicative of the risk of the child for autism spectrum disorder, by the convolutional neural network, based on the facial keypoints extracted from the video images of the child and the movement features derived from the video images of the child.

12. The method of claim 11, wherein the movement features include a weight feature indicative of intensity of perceived force in the movement, a space feature indicative of distance of the arms of the child relative to the body of the child, and a time feature indicative of a change in tempo in the movement.

13. The method of claim 11, wherein the convolutional neural network includes two one-dimensional convolution layers to identify temporal data patterns, three dense layers for classification, and a plurality of dropout layers to avoid overfitting.

14. The method of claim 11, further comprising:
mimicking the expression of human emotion by an emotionally expressive robot.

15. The method of claim 14, wherein the emotionally expressive robot comprises a humanoid robot programmed to mimic the expression of human emotion through gestures or speech or a facially expressive robot programmed to mimic the expression of human emotion through facial expression.

16. The method of claim 14, wherein the video images are captured while the child interacts with the emotionally expressive robot.

17. The method of claim 14, further comprising:
providing sensory stimulation by each of a plurality of sensory stations.

18. The method of claim 17, wherein the plurality of sensory stations include a seeing station that provides visual stimulus, a hearing station that provides auditory stimulus, a smelling station provide olfactory stimulus, a tasting station that provides gustatory stimulus, or a touching station that provides tactile stimulus.

19. The method of claim 17, wherein the video images are captured while the child observes the emotionally expressive robot interacting with each of the sensory stations.

20. Non-transitory computer readable storage media storing instructions that, when executed by a hardware computer processor, cause a computer to determine whether a child may be at risk for autism spectrum disorder based on movement and facial expression by:
receiving video images of the child;
extracting body tracking keypoints and facial keypoints from the video images;
deriving movement features from the body tracking keypoints;
providing the movement features and body tracking keypoints extracted from the video images to a convolutional neural network trained on a dataset that includes movement features and body tracking keypoints of children diagnosed with autism spectrum disorder; and
generating a diagnosis indicative of the risk for autism spectrum disorder by the convolutional neural network.
